(19) United States
(12) Patent Application Publication
(10) Pub. No.: US 2010/0221706 A1
(43) Pub. Date: Sep. 2, 2010

(54) KIT FOR THE AMPLIFICATION OF NUCLEIC ACIDS

(75) Inventors: Maurizio Gramegna, Cigognola (Pavia) (IT); Roberta Tagliabue, Lainate (Milano) (IT); Dario Russo, Ceran (Novara) (IT)

Correspondence Address: Steinfl & Bruno, 301 N Lake Ave Ste 810, Pasadena, CA, 91101, US

(73) Assignee: CLONIT S.r.l., Milano (IT)

(21) Appl. No.: 12/162,472

(22) PCT Filed: Jan. 29, 2007
(86) PCT No.: PCT/IB2007/050294

§ 371 Date: Jul. 28, 2008

(30) Foreign Application Priority Data
Jan. 31, 2006 (IT) TO2006A000063

(51) Int. Cl. C12Q 1/68

(52) U.S. Cl. 435/6

(57) ABSTRACT

A ready-to-use kit for the PCR amplification of nucleic acids is described, comprising at least one vessel into which a master mix comprising the salts, the buffer and the deoxynucleotide triphosphates required for the PCR amplification is prealiquotted, the said master mix being mixed with an effective amount of an inert temperature controllable polymer such that the final mixture thus formed is in the liquid phase at a predetermined temperature and in the gel phase at a temperature lower than a second predetermined temperature.

The present invention relates in general to a kit for the amplification of nucleic acids. More specifically, the invention concerns a kit for the amplification of nucleic acids which are viral, genomic, synthetic or of another nature by means of Real Time PCR technology.

The molecular biology techniques designated as NAT (Nucleic Acid Technology), in other words amplification of nucleic acids, have undergone very considerable development in the last twenty years, so as to be currently regarded as reference techniques for diagnostics in many sectors such as viral, bacterial, and protozoan, right down to genetic diseases and forensic medicine.

In addition to the qualitative techniques for the identification of pathogens, methods for quantification of nucleic acids have recently been developed, and are arousing ever greater interest. The first application of these techniques took place in the viral field for the quantification of the HCV virus, significant for the treatment and the follow up of the patient.

The most widespread nucleic acid amplification technique is based on a method designated as the polymerase chain reaction (PCR) which, in its most recent modes of implementation (Real Time PCR), makes use of probes labelled with fluorescent molecules. In short, the PCR method is based on the use of specific oligonucleotide primers for the reaction of a thermally stable polymerase such as for example Taq polymerase (Thermus aquaticus) or Tth polymerase (Thermus thermophilus), on a DNA template.

Where it is necessary to start from an RNA template (for example in the case of RNA viruses such as HCV, HIV or SARS), it is necessary to perform an initial reverse transcription step using a reverse transcriptase (for example AMV RT or MoMULV) in order to obtain a hybrid double helix made up of one strand of RNA and one strand of cDNA. In the case of RNA, enzymes such as the said Tth polymerase which have the double activity of reverse transcription and polymer-isation can also be used.

The repetition of cycles comprising a denaturation phase, in which the two helices of the DNA are separated, an annealing phase in which the primers hybridise specifically to complementary sequences on the target DNA (the so-called target sequences) and an elongation phase, in which the thermally stable polymerase synthesises a copy molecule on the template of the target DNA, makes it possible to secure a very considerable enrichment of the starting target DNA.

In a particularly widespread implementation mode of the PCR method, called Real Time PCR, the determination of the quantity of nucleic acid present in a sample is effected while the reaction is in progress, in other words indeed “in real time”. The principle of Real Time PCR is based, as mentioned above, on the detection and the quantification of the fluorescent signal which develops during the amplification reaction when the sample is positive for the pathogen sought. In this reaction, probes specific for the sequences identified by the primers of the amplification reaction are used, the said probes being labelled with a “reporter” molecule and a “quencher”. When the probe is intact, the quencher is located in the vicinity of the reporter and this causes the suppression of the fluorescent signal which the reporter is capable of emitting. During the amplification reaction, the probe, which hybridises due to homology within the target sequence for the amplification, is cut by the polymerase enzyme separating the quencher from the reporter which, no longer being suppressed by the quencher, emits a detectable fluorescent signal, the intensity whereof is directly proportional to the number of amplified copies of the target present in the mixture.

Normally, the Real Time PCR reaction is performed using 96-well plates which are placed in the slots of a thermocycler instrument to be processed. The number of samples analysed per plate is variable and generally is around 30-40, since the PCR reaction is often carried out with duplicates.

The reaction mixtures used in the PCR method are generally in the liquid phase, which causes problems connected with the possibility of transferring solution from one well to another during the preparation phase and also of carry-over due to evaporation of the solution in the actual reaction phase.

In order to confront these problems, the Italian patent application RM1997A000038 describes a process for the amplification of nucleic acids wherein at least one of the oligonucleotide primers is mixed with an inert thermally controllable polymer such that the mixture formed is in the liquid phase at a predetermined temperature and in the gel phase at a lower temperature, preferably around 37° C.

One of the main problems which nonetheless remain to be overcome for the application of the PCR on a large scale as a diagnostic assay consists in the difficulty of the method, which thus requires specialised staff. The operator must in fact prepare in advance all the reagents necessary for setting up the PCR reaction and aliquot into each of the wells of the plate the correct volume of amplification mixture, with all the risks deriving from the variability due to the operator.

For the purpose of overcoming these disadvantages, the subject-matter of the invention is a ready-to-use kit for the PCR amplification of nucleic acids, comprising at least one vessel into which a master mix for the PCR amplification of the target nucleic acid is prealiquotted, the said master mix comprising the salts, the buffers and the deoxynucleotide triphosphates required for the PCR amplification, the said master mix being mixed with an effective amount of an inert temperature controllable polymer such that the final mixture thus formed is in the liquid phase at a predetermined temperature and in the gel phase at a temperature lower than a second predetermined temperature. The said second predetermined temperature is preferably about 37° C.

The term “master mix” refers to a reaction mixture, generally in the form of an aqueous solution, comprising all or part of the reagents required for the PCR amplification in the appropriate quantities. In this first embodiment, the master mix comprises the salts and the buffers required for the activity of the polymerase enzyme and the deoxynucleotide triphosphates (dNTPs).

The term “prealiquotted” indicates that the master mix and the components thereof are provided inside the reaction vessel in predetermined effective concentrations so that the amplification of the target nucleic acid sequence can take place.

In a second embodiment of the kit according to the invention, the master mix further comprises the oligonucleotide primers required for the PCR amplification of the target nucleotide sequence.

On account of its characteristics, the kit of the invention makes it possible advantageously to simplify the operations necessary for the performance of the PCR method and to reduce the test times, and also to overcome the problem of the variability due to the operator. These characteristics render the kit of the invention particularly suitable for use for clinical applications of the PCR on a large scale, even by non-specialist staff.

The vessel of the kit can be for example a test-tube, a vial or a plate of any size and configuration. Among plates, dismantlable ones are preferred. As an alternative to the embodiment wherein the kit comprises a single vessel, it is also envisaged that the kit may include a plurality of vessels—generally test-tubes or vials, connected in series or as a ring.

The inert temperature controllable polymer used in the kit of the invention is a polymer which exists in a “gel” state at ambient temperature, and in any case at a temperature below a second predetermined temperature (preferably about 37° C.), and in a “sol” state after appropriate heating, then to return to the “gel” state when the temperature again approaches ambient temperature. When the system is in the solid (“gel”) state, the kinetics are essentially reduced to zero, which makes it possible to avoid the formation of dimers of primers.

According to a third embodiment of the kit according to the invention, the master mix, which in addition to the already mentioned oligonucleotide primers and reagents necessary for the PCR reaction (salts, buffers, dNTPs) also comprises the enzymes necessary for the amplification of the target nucleic acid, is maintained in the gel state (and therefore in a mixture with the temperature controllable polymer).

The enzymes required for the amplification of a target DNA sequence by PCR are polymerase enzymes. A preferred polymerase for use in the kit of the invention is Taq polymerase. In the event that the target sequence is a molecule of RNA, a reverse transcriptase enzyme is also required. Alternatively, an enzyme having both polymerase activity and reverse transcriptase activity, such as the Tth polymerase already mentioned, can be used.

In a further embodiment of the kit according to the invention, the master mix also comprises a labelled probe, preferably fluorescent, for the real time detection of the amplification products and possibly an internal DNA or RNA control. In this case, the master mix may also comprise primers and probes which are specific for the amplification and detection of the internal control.

In yet another embodiment of the kit of the invention, the master mix also comprises an effective amount of one or more inert dyes, preferably methylene blue or malachite green.

If the kit is intended for the amplification of a plurality of different target nucleotide sequences, the use of a different dye for each target nucleic acid sequence to be amplified can be envisaged, as well as the use of primers and probes specific for the amplification and the detection of each target nucleic acid sequence.

An example of an inert temperature controllable polymer suitable for use in the kit according to the invention is the polysaccharide agarose, preferably at a concentration comprised within the range 0.5-0.8%, but the skilled in the art will realise that any inert temperature controllable polymer with a transition from sol to gel at a predetermined temperature can equally be used. When the ambient temperature is approached, the solution passes into the gel state with considerable advantages both for storage (even at ambient temperature) and for manipulation.

As an alternative to the use of a single temperature controllable polymer, the use of different polymers with different temperature control properties can also be envisaged, to be combined even within a single reaction test-tube or well. Such a kit is particularly indicated for performing both the reverse transcription and the polymerisation in a single reaction test-tube or well by operating exclusively on the thermal reaction protocol which takes account of the different melting temperatures of the polymers.

The kit of the invention can be used for the PCR amplification of any nucleic acid, of viral, bacterial, animal or plant origin and finds applications in diagnostics, in genetics, in oncology and in forensic medicine. The viral nucleic acids include for example those from the following viruses: HBV, HCV, SARS, CMV, HPV, HSV and HTLV I and II.

The examples that follow are provided exclusively for illustrative purposes and are not intended to limit the scope of the invention as defined in the appended claims.

### EXAMPLES

### Universal Solid Real Time Master Mix

Universal Solid Real Time Master Mix is a universal master mix for systems in real time PCR based on Taqman Probes. The principal characteristic of this system is that it has been developed as a solid by the use of thermopolymers designed to effect the change from the solid state to the liquid at a desired temperature, without interfering with the detection of the fluorescent signal or with the amplification reaction. Universal Solid Real Time Master Mix is prepared in dismantlable plates so as to allow the operator to carry out the analysis of a variable number of samples depending on the various requirements and contains, already mixed, all the components necessary for a quantitative Real Time PCR reaction except for the template DNA, the primers, the probes and the Taq polymerase.

Thus, in a total volume of 25 μl, every amplification reaction will be prepared in the following way:

### R. Time Solid Master Mix

| | | |
|---|---|---|
| Forward primer | 500 | nM |
| Reverse primer | 500 | nM |
| Probes | 200 | nM |
| Taq Gold (Applied Biosystems) | 0.05 | U/μl |

To the thus constituted amplification mixture are added 5 μl of DNA from the sample concerned and the reaction is performed in accordance with the following amplification programme:

### Amplification Programme

| | | | | 45 cycles | | | |
|---|---|---|---|---|---|---|---|
| 50° C. | 2 mins | 95° C. | 10 mins | 95° C. | 15 secs | 60° C. | 1 min |

Detection system used: ABI-PRISM 7000 DETECTION SYSTEM.

### Composition of Universal Solid Real Time Master Mix:

| Reagents | Concentration | |
|---|---|---|
| TAQ buffer | 1 | x |
| MgCl₂ | 3.5 | mM |
| dNTPs | 200 | μM |
| Reference dye (ROX) | 1 | x |

| | |
|---|---|
| Thermopolymer (agarose) | 0.8% |

Universal Solid Real Time Master Mix was checked and compared with Clonit systems already used for the quantitative analysis of some specific targets.

### Quantitative Analysis of the HCV Virus

The following table summarises the concentrations and the volumes necessary for completion of the Real Time reaction mixture.

| | Initial | | Qty. | | Qty. | | Final | |
|---|---|---|---|---|---|---|---|---|
| Reagents | conc. | | 1 test | | 10 tests | | Conc. | |
| Forward | 100 | μM | 0.225 | μl | 2.25 | μl | 900 | nM |
| primer | | | | | | | | |
| Reverse | 100 | μM | 0.125 | μl | 1.25 | μl | 500 | nM |
| primer | | | | | | | | |
| Probes | 10 | μM | 0.5 | μl | 5 | μl | 200 | nM |
| Taq GOLD | 5 | U/μl | 0.25 | μl | 2.5 | μl | 0.05 | U/μl |
| DEPC H₂O | | | 0.9 | μl | 9 | μl | | |
| | | | 2 | μl | 20 | μl | | |

Amplification with Universal Solid Real Time Master Mix

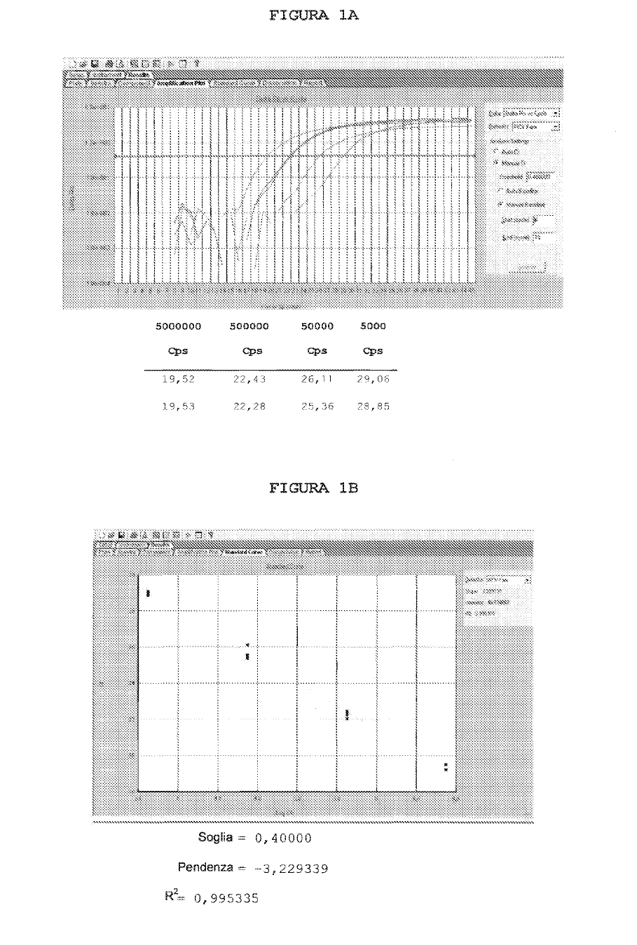

During the amplification, i.e. “in real time”, an amplification graph is displayed on the screen of the detection instrument where the progress of the amplification can be followed directly (see FIG. 1A). The earlier the signal grows, the higher is the concentration of target nucleic acid in the sample under test.

The software also constructs a straight line (see FIG. 1B) whose gradient is an indicator of the good linearity of the standards present in the analytical session and makes it possible to validate the session.

The controls used are secondary standards, labelled EC, which serve as a reference for verifying the method in question. The concentrations of these are maintained at a dilution factor of ten one from another. The graph shows how the amplification curves match with absolute fidelity the concentrations attributed to the standards themselves, verifying and validating the system.

Linearity and Proportionality of the System

These parameters express the capacity of an analytical method to obtain results directly proportional to the concentration of the analyte in the sample. Linearity and proportionality are verified with standard solutions at different concentration levels. This function must be found to be linear and its most characteristic manifestation is a correlation coefficient very close to the value 1.

TABLE 1

| | | Standard | | Standard | | |
|---|---|---|---|---|---|---|
| R² | Mean | Deviation | N | Error | UCL | ICL |
| 0.9951 | 0.9965 | 0.00303 | 4 | 0.0015 | 0.998 | 0.994686 |
| 0.9988 | | | | | | |
| 0.9992 | | | | | | |
| 0.9929 | | | | | | |

LOQ (Limit of Quantitation)-LOD (Limit of Detection)

LOQ (limit of quantitation): expresses the smallest quantity of analyte in a sample which can be determined and quantified, with an acceptable precision and accuracy value. LOQ can be evaluated as a function of the standard deviation relative to the gradient of the calibration curve.
LOD (limit of detection): expresses the smallest quantity of analyte which can be detected but not necessarily quantified. LOD can be evaluated as a function of the standard deviation relative to the gradient of the calibration curve.

TABLE 2

| | | | St. | | | |
|---|---|---|---|---|---|---|
| | | | Dev. | | | |
| | | Variance | of | LOG | | |
| Gradient | Mean | of mean | mean | (LOQ) | LOQ [cps] | LOD (cps) |
| −3.21 | −3.29 | 0.27794 | 0.5272 | 1.5993 | 39.75 | 13.248 |
| −3.21 | | | | | | |
| −3.44 | | | | | | |
| −3.32 | | | | | | |

Comparison Between Universal Solid Real Time Master Mix and HCV RNA Real Time Master Mix (Mix in Liquid)

In order to verify the neutrality of the polymer used to make the master mix which is the object of the present patent, this was compared with a mixture for Real Time PCR in the normal version in liquid. The results are totally superimposable and thus validate the system.

### Table 3

TABLE 3

| HCV-RNA REAL | | | | UNIVERSAL SOLID REAL | | | |
|---|---|---|---|---|---|---|---|
| TIME MASTER MIX | | | | TIME MASTER MIX | | | |
| 5000000 cps | 500000 cps | 50000 cps | 5000 cps | 5000000 cps | 500000 cps | 50000 cps | 5000 cps |

(Cycles for exit of signal)

| | | | | | | | |
|---|---|---|---|---|---|---|---|
| 19.5 | 23.375 | 27.09 | 30.65 | 19.53 | 22.36 | 25.74 | 28.96 |

| | |
|---|---|
| Gradient −3.7255 | Gradient −3.212 |
| R² 0.9996 | R² 0.9994 |

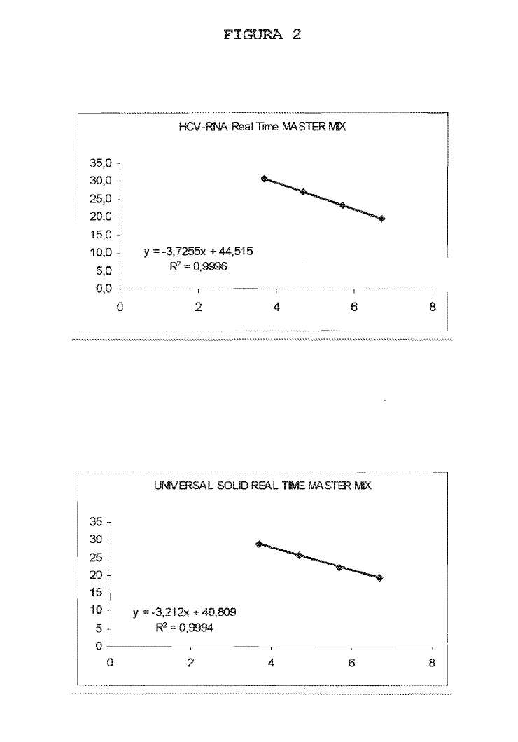

FIG. 2 shows the graphs of the gradients of the curves obtained.

### Quantitative Analysis of the HBV Virus

The following table summarises the concentrations and the volumes necessary for completing the Real Time reaction mixture.

TABLE 4

| | Initial | | Qty. | | Qty. | | Final | |
|---|---|---|---|---|---|---|---|---|
| Reagents | conc. | | 1 test | | 10 tests | | Conc. | |
| Forward | 100 | μM | 0.125 | μl | 1.25 | μl | 900 | nM |
| primer | | | | | | | | |
| Reverse | 100 | μM | 0.125 | μl | 1.25 | μl | 500 | nM |
| primer | | | | | | | | |
| Probes | 10 | μM | 0.1 | μl | 1 | μl | 200 | nM |
| Taq GOLD | 5 | U/μl | 0.25 | μl | 2.5 | μl | 0.05 | U/μl |
| DEPC H₂O | | | 1.4 | μl | 14 | μl | | |
| | | | 2 | μl | 20 | μl | | |

Amplification with Universal Solid Real Time Master Mix

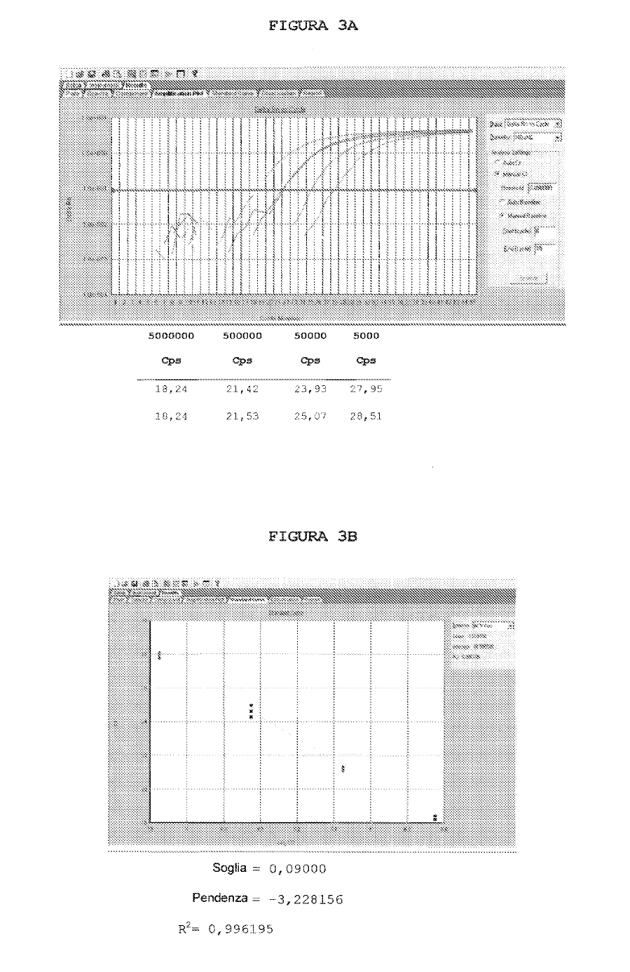

FIG. 3A is a graph which shows the progress of the amplification. The gradient of the straight line in FIG. 3B is an indicator of the good linearity of the standards present in the analytical session and makes it possible to validate the session.

Linearity and Proportionality of the System

TABLE 5

| | | Standard | | Standard | | |
|---|---|---|---|---|---|---|
| R² | Mean | Deviation | N | Error | UCL | ICL |
| 0.9991 | 0.98467 | 0.01964 | 3 | 0.0113 | 0.996 | 0.973327 |
| 0.9926 | | | | | | |
| 0.9623 | | | | | | |

LOQ (Limit of Quantitation)-LOD (Limit of Detection)

TABLE 6

| | | Variance | St. Dev. | LOG | LOQ | LOD |
|---|---|---|---|---|---|---|
| Gradient | Mean | of mean | of mean | (LOQ) | [cps] | (cps) |
| −3.2395 | −3.40343 | 0.65952 | 0.65952 | 1.9378 | 86.66 | 28.886 |
| −3.3863 | | | | | | |
| −3.5845 | | | | | | |

Comparison Between Universal Solid Real Time Master Mix and HBV RNA Real Time Master Mix (Mix in Liquid)

TABLE 7

| HBV-RNA REAL | | | | UNIVERSAL SOLID REAL | | | |
|---|---|---|---|---|---|---|---|
| TIME MASTER MIX | | | | TIME MASTER MIX | | | |
| 5000000 cps | 500000 cps | 50000 cps | 5000 cps | 5000000 cps | 500000 cps | 50000 cps | 5000 cps |
| 18.85 | 21.63 | 25.59 | 28.80 | 18.24 | 21.475 | 24.5 | 28.03 |

| | |
|---|---|
| Gradient −3.381 | Gradient −3.2395 |
| R² 0.9959 | R² 0.9991 |

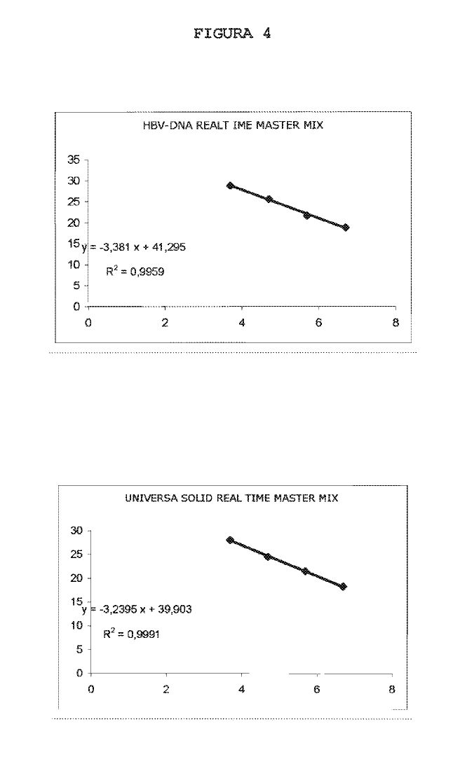

FIG. 4 shows the graphs of the gradients of the curves obtained.

1. A ready-to-use kit for the PCR amplification of nucleic acids, the kit comprising at least one vessel into which a master mix comprising salts, buffers and deoxynucleotide triphosphates for the PCR amplification is prealiquotted, said master mix being mixed with an effective amount of an inert temperature controllable polymer such that the final mixture thus formed is in a liquid phase at a first predetermined temperature and in a gel phase at a temperature lower than a second predetermined temperature.

2. The kit according to claim 1, wherein the master mix further comprises oligonucleotide primers for amplification of a target nucleotide sequence.

3. The kit according to claim 1, wherein the master mix further comprises an enzyme for amplification of a target nucleotide sequence.

4. The kit according to claim 3, wherein the said enzyme has polymerase activity and/or reverse transcriptase activity.

5. The kit according to claim 4, wherein the master mix comprises an enzyme having polymerase activity and an enzyme having reverse transcriptase activity and wherein the master mix is mixed with an effective amount of a plurality of different temperature controllable polymers.

6. The kit according to claim 1, wherein the master mix further comprises a labelled probe for detection of amplification products of a target nucleotide sequence.

7. The kit according to claim 1, further comprising an internal DNA or RNA control.

8. The kit according to claim 7, further comprising primers for amplification of the internal DNA or RNA control.

9. The kit according to claim 7, further comprising a labelled probe for detection of amplification products of the internal DNA or RNA control.

10. The kit according to claim 1, for amplification of a plurality of different target nucleotide sequences, wherein the master mix comprises primers specific for each of said plurality of different target nucleotide sequences.

11. The kit according to claim 1, wherein the master mix further comprises an effective amount of one or more inert dyes.

12. The kit according to claim 6, wherein the labelled probe is fluorescent.

13. The kit according to claim 1, wherein the at least one vessel is selected from the group consisting of test-tubes, vials and plates of any size and configuration.

14. The kit according to claim 13, wherein the vessel is a dismantlable plate.

15. The kit according to claim 13, comprising a plurality of test-tubes or vials connected in series or as a ring.

16. The kit according to claim 1, wherein the second predetermined temperature is about 37° C.

17. The kit according to claim 1, wherein the inert temperature controllable polymer is agarose.

18. The kit according to claim 10, wherein said master mix further comprises labelled probes specific for each of said plurality of different target nucleotide sequences.
